(19) United States
(12) Patent Application Publication
(10) Pub. No.: US 2021/0236427 A1
(43) Pub. Date: Aug. 5, 2021

(54) Development of Lipid Matrix Granules with Incorporation of Polysaccharides for Effective Delivery of an Antimicrobial Essential Oil

(71) Applicant: University of Manitoba, Winnipeg (CA)

(72) Inventors: Chengbo Yang, Winnipeg (CA); Song Liu, Winnipeg (CA); Martin Nyachoti, Winnipeg (CA)

(21) Appl. No.: 17/052,022

(22) PCT Filed: May 6, 2019
(86) PCT No.: PCT/CA2019/050599

§ 371 Date: Oct. 30, 2020

Related U.S. Application Data
(60) Provisional application No. 62/667,016, filed on May 4, 2018.

(51) Int. Cl. A61K 9/16; A61K 31/05; A23K 20/163; A23K 20/158

(52) U.S. Cl. CPC A61K 9/1652; A61K 9/1617; A23K 20/158; A23K 20/163; A61K 31/05

(57) ABSTRACT

Antibiotics have long been used at sub-therapeutic levels to control incidences of post-weaning diarrhea and to improve growth performance in pigs. However, the current trend world-wide is to eliminate the use of in-feed antibiotics due to increased public concerns over the spread of antibiotic resistance in bacterial pathogens, which poses a threat to public health. Alternatives to in-feed antibiotics are needed. Thymol essential oil exhibits strong in vitro antibacterial activity; however; direct inclusion of essential oils to pig feeds has limited efficacy due to their high volatility, low stability during feed processing, interactions with other feed components and poor availability in lower gut. To solve these problems, we developed lipid matrix beads using thymol and a fatty acid with incorporation of 2% polysaccharides via a melt-granulation technique. Laurie acid was identified as a suitable carrier for thymol. In vitro release of thymol from lipid matrix granules was determined using simulated salivary fluid (SSF), simulated gastric fluid (SGF) and simulated intestinal fluid (SIF), respectively. The lipid matrix granules with 2% polysaccharides exhibited a slow release rate (%) of essential oil and fatty acid in SSF (21.2±2.3; 36±1.1), SGF (73.7±6.9; 54.8±1.7) and SIF (99.1±1.2; 99.1±0.6), respectively. However, the lipid matrix granules without polysaccharides had quick release (%) of essential oil and fatty acid from the SSF (79.9±11.8; 84.9±9.4), SGF (92.5±3.5; 75.8±5.9) and SIF (93.3±9.4; 93.3±4.6), respectively.

### PRIOR APPLICATION INFORMATION

The instant application claims the benefit of U.S. Provisional Patent Application 62/667,016, filed May 4, 2018 and entitled “DEVELOPMENT OF LIPID MATRIX GRANULES WITH INCORPORATION OF POLYSACCHARIDES FOR EFFECTIVE DELIVERY OF AN ANTIMICROBIAL ESSENTIAL OIL”, the entire contents of which are incorporated herein by reference for all purposes.

### BACKGROUND OF THE INVENTION

Young animals are most vulnerable to diseases, and antimicrobials are widely used in livestock production to maintain health and productivity. Global consumption of antimicrobials in food animal production was estimated at 63,151 tons in 2010 and the annual consumption of antimicrobials per kilogram of animal produced is 148 mg/kg and 172 mg/kg for chicken and pigs, respectively (Van Boeckel et al., 2015). This practice may lead to the spread of antimicrobial-resistant pathogens in both livestock and humans, posing a significant public health threat (Yang et al., 2015). A ban against using antibiotic growth promoters in food animal production has been implemented in the European Union countries since 2006 (Bengtsson and Wierup, 2006), both Health Canada and the U.S. Food and Drug Administration placed restrictions on antibiotic use in animals in December 2016, and more countries are expected to follow. There are a number of challenges after the withdrawal of antibiotics from feed (Zhao et al., 2007). The cost-effectiveness of substituting antibiotics with alternatives is the most challenging one, which remains critical for ensuring long-term sustainable animal production (Yang et al., 2015). Organic acids (Eckel et al., 1992; de Lange et al., 2010), enzymes (Bedford and Cowieson, 2012; Kiarie et al., 2013), probiotics (Musa et al., 2009; Heo et al., 2013), antimicrobial peptides (Choi et al., 2013) and essential oils (Windisch et al., 2008; Randrianarivelo et al., 2010; Gong et al., 2014) have been widely recognized as potential alternatives to in-feed antibiotics. Essential oils are defined as plant-derived natural bioactive compounds with positive effects on animal growth and health (Puvaca et al., 2013). Essential oils are known to have antimicrobial and antioxidative properties (Brenes et al., 2010), and traditionally have been used as complementary or alternative medicines to improve human health or cure human diseases (Kim et al., 2008). With the identification of active components in plant extracts and some progress in mechanistic studies of these components in animals, there have been increased research efforts to use essential oils as substitutes for antibiotics in animal diets (Li et al., 2012). The application of essential oils in feed has been based on antimicrobial effects and immunity regulation. However, the minimum inhibitory concentration of most essential oils is significantly higher than the levels that are acceptable in animal production from the viewpoint of cost-effectiveness and feed palatability (Yang et al., 2015). Therefore, it is vital to investigate the specific effect and target site (either animal host or its microbiota) of individual essential oils, which will facilitate the application of essential oils in feed.

Essential oils are very volatile and can evaporate rapidly, leading to varied final concentrations in end products (Lambert et al., 2001). The stability of essential oils during feed processing is often questionable. In addition, several studies indicated that carvacrol, thymol, eugenol and trans-cinnamaldehyde were mainly or almost completely absorbed in the stomach and the proximal small intestine of piglets after oral intake (Michiels et al., 2008). Additionally, essential oils may be absorbed into feed components, leading to a reduced antimicrobial activity (Si et al., 2006). Therefore, without proper protection, most essential oils will be lost during feed processing and delivery to the pig gut and thus may not be able to reach the lower gut of pigs where most pathogens reside and propagate. This will reduce the profitability of feed mills and is one of major barriers for essential oil application in feed. Thus, it is crucial to develop an effective and practical delivery method for using essential oils in feeds.

Microencapsulation has become one of the most popular methods to deliver essential oils into the lower gut (Piva et al. 2007; Chitprasert et al. 2014). An ideal microencapsulation should not only stabilize essential oils, but also release them specifically in the target regions of intestine (Chen et al. 2017). Many materials including polysaccharides (alginate xanthan gum), proteins (whey protein and gelatin) and lipids (milk fat and hydrogenated fat) have been used to encapsulate essential oils for effective delivery in the gut (Piva et al. 2007; Zhang et al. 2016; Chen et al. 2017). Lipid is the most commonly used material for encapsulating essential oils in feed applications. However, there are still challenges to fully protect and deliver essential oil into the lower gut, which lead to inconsistent results. The potential reasons include: 1) general drawbacks of microencapsulation, such as, for example, the high manufacturing costs, low particle strength (which results in a low stomach bypass rate), the fact that some enteric coating chemicals cannot be used in animal feeds; and premature melting of the lipid matrix at elevated temperatures during transportation, storage, feed processing or consumption (i.e. body temperature); 2) limited knowledge on the morphology and microstructure of lipid microparticles; and 3) no in-depth studies to elucidate the mechanisms underlying the phenomenon of stability or release of essential oils from the lipid microparticles.

A viable alternative to in-feed antibiotics needs to be safe to the public, cost-effective in production and friendly to the environment (Gong et al., 2014). Because of these multiple requirements, no single alternative has been developed that can fully replace antibiotics in feed.

Medium chain fatty acids (MCFAs), including lauric acid (C12), capric acid (C10), caprylic acid (C8), carboxylic acids (C7 and C9) and caproic acid (C6) and their derivatives, are another type of alternative to antibiotics for piglets (Boyen et al., 2008; Zentek et al., 2011, 2012; Hanczakowska et al., 2013). MCFAs has the capacity to fight against microbial activity of Salmonella and E. coli (Dierick et al., 2002; Rossi et al., 2010). Research carried out by Han et al. (2011) shows that the performance of pigs fed eucalyptus MCFA blend was the same as that of antibiotics. MCFAs are shown to have a good antimicrobial effect on both G⁻ and G⁺ bacteria. The effectiveness of the antimicrobial activity of MCFA towards some groups of bacteria is different based on their chain lengths (Rossi et al., 2010). Caprylic acid may have a similar mode of action as short chain fatty acids; that is, MCFAs may inactivate bacteria by creating an acidic environment or by having a direct impact on the expression of virulence factors necessary for Salmonella colonization. At low dietary levels, MCFAs may be regarded as modulators of the gastric microbiota in weaned piglets. When treated with a combination of oregano oil and caprylic acid, additive effects were observed with multiple strains of Salmonella, Listeria monocytogenes, E. coli and Streptococcus aureaus (Hulenkove and Borilovs, 2011). Similar effects of cinnamaldehyde and lauric acid against Brachyspira hyodysenteriae, the causative pathogen of swine dysentery, were observed in vitro (Maele et al., 2016).

MCFAs are generally recognized as safe (GRAS) by the Food and Drug Administration (de Los Santos et al., 2008). However, some MCFA and their derivatives have strong and unpleasant smells that can reduce feed palatability and feed intake of pigs (Zentek et al., 2011). These may be overcome by using a combination of essential oils and MCFAs. However, there is no information on the in vivo application of the combination of essential oils and MCFAs in swine production.

### SUMMARY OF THE INVENTION

According to a first aspect of the invention, there is provided a granulated particle comprising:

an essential oil;

a fatty acid or glyceride of a fatty acid;

a starch source; and

a polysaccharide.

According to another aspect of the invention, there is provided a method of preparing a granulated particle comprising:

mixing an essential oil and a fatty acid;

adding a starch source to the mixture and mixing;

adding polysaccharide to the mixture;

allowing solid particles to form; and

granulating the particles.

### BRIEF DESCRIPTION OF THE DRAWINGS

FIG. 1. a) Pictures showing the molten mixture of thymol and fatty acids were placed at room temperature (23° C.) immediately (a) and at 6 h (b). FA1—mixture of thymol and lauric acid, FA2—mixture of thymol and palmitic acid; FA3—mixture of thymol and stearic acid at 0 min and 6 h set at room temperature (23° C.).

FIG. 2. Morphology of thymol and lauric acid before crystallization and after crystallization observed with a light microscope.

FIG. 3. Morphology of lipid matrix granules with/without 2% polysaccharide solution observed with a light microscope.

FIG. 4. Surface diagram of lipid matrix granules with/without 2% polysaccharide solution observed with a light microscope.

FIG. 5. In vitro release profile of thymol and lauric acids from lipid matrix granules with 2% polysaccharide solution using simulated fluids (SSF— simulated salivary fluid, SGF—simulated gastric fluid and SIF— simulated intestinal fluid). (Mean±SD, n=4).

FIG. 6. In vitro release profile of thymol and lauric acid from lipid matrix granules without 2% polysaccharide solution using simulated fluids (SSF— simulated salivary fluid, SGF—simulated gastric fluid and SIF— simulated intestinal fluid). (Mean±SD, n=4).

FIG. 7. Stability of lipid matrix granules with/without 2% polysaccharide solution stored at 4° C. for 12 weeks. (Mean±SD, n=4).

FIG. 8. Stability of lipid matrix granules with/without 2% polysaccharide solution stored at room temperature (23° C.) for 2 weeks. (Mean±SD, n=4).

FIG. 9. A scanning electron microscope (SEM) diagram (500, 1,000, 3,000 and 5,000 fold) of lipid matrix granules prepared with 2% polysaccharide solution.

FIG. 10. Differential scanning calorimetry (DSC) of (A) Thymol, (B) Lauric acid, and (C) Mixture of thymol and lauric acid (50:50 wt %). 2nd run with heating rate 10° C./min from −10° C. to 80° C.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Antibiotics have long been used at sub-therapeutic levels to control incidences of post-weaning diarrhea and to improve growth performance in pigs. However, the current trend world-wide is to eliminate the use of in-feed antibiotics due to increased public concerns over the spread of antibiotic resistance in bacterial pathogens, which poses a threat to public health. Alternatives to in-feed antibiotics are needed.

Thymol essential oil exhibits strong in vitro antibacterial activity; however; direct inclusion of essential oils to pig feeds has limited efficacy due to their high volatility, low stability during feed processing, interactions with other feed components and poor availability in the lower gut.

To solve these problems, we developed lipid matrix granules using thymol and a fatty acid with incorporation of 1-5% polysaccharides solution via a melt-granulation technique. Lauric acid was identified as a suitable carrier for thymol.

In vitro release of thymol from lipid matrix granules was determined using simulated salivary fluid (SSF), simulated gastric fluid (SGF) and simulated intestinal fluid (SIF), respectively, as described below. The lipid matrix granules with 2% polysaccharides solution exhibited a slow release rate (%) of essential oil and fatty acid in SSF (21.2±2.3; 36 1.1), SGF (73.7±6.9; 54.8±1.7) and SIF (99.1±1.2; 99.1±0.6), respectively. However, the lipid matrix granules without polysaccharides had quick release (%) of essential oil and fatty acid from the SSF (79.9±11.8; 84.9±9.4), SGF (92.5±3.5; 75.8±5.9) and SIF (93.3±9.4; 93.3±4.6), respectively. The lipid matrix granules with or without polysaccharides both had good stability (>90%) after being stored at 4° C. for 12 weeks or at room temperature (23° C.) for 2 weeks.

The results demonstrated that the method can be used to deliver essential oils to the pig intestinal tract effectively, as discussed below.

According to an aspect of the invention, there is provided a granulated particle comprising: an essential oil, a fatty acid, a starch source and a polysaccharide.

According to an aspect of the invention, there is provided a granulated particle comprising: at least one essential oil, at least one fatty acid, at least one starch source and at least one polysaccharide.

The fatty acid may be any suitable saturated fatty acid or glyceride thereof that is compatible with and suitable for use in animal feeds.

In some embodiments, the fatty acid is a fatty acid that is solid at room temperature but that when mixed with an essential oil, for example, an essential oil of interest, the mixture of the selected fatty acid and the essential oil of interest forms a liquid, for example, a homogeneous liquid or liquid phase.

In some embodiments, the fatty acid is a medium chain fatty acid, as discussed herein.

In some embodiments, the fatty acid is selected from the group consisting of monolaurin, caprylic acid, triglycerides of caprylic acid, capric acid, triglycerides of capric acid, and lauric acid.

In some embodiments, the essential oil is selected from the group consisting of carvacrol, cinnamaldehyde, thymol, citral, curcumin, allicin and capsaicin.

In some embodiments, the essential oil is thymol, carvacrol or cinnamaldehyde and the fatty acid is lauric acid.

In some embodiments, the essential oil is thymol and the fatty acid is lauric acid.

Starch is generally recognized as safe and has been widely used in the food industry for microencapsulation because it is biodegradable, edible, commonly available, abundant, low cost, nonallergic, easy to use and thermo-processable. In addition, the use of combinations of starches for example corn-starch and pre-gelatinized starch influences water retention. Pre-gelatinized starch is starch that has undergone processing under intense heat conditions by cooking, drying and making into a fine powder, causing it to be cold-water soluble starch. Pregelatinized starch is more stable, durable, less soluble, viscous and increases digestibility due to the denaturation of protein and the rupture of hydrogen bonds during processing.

Specifically, as discussed herein, thymol and lauric acid are very compatible. While not wishing to be bound to a particular theory or hypothesis, it is believed that this compatibility is due to the fact that thymol and lauric acid have a similar melting point. Alternatively, thymol may form hydrogen bonds with lauric acid more easily than with other fatty acids with more carbons. It is also possible that the mixture of thymol and lauric acid slows down the crystallization rate.

The polysaccharides can be any polysaccharide that contains a carboxyl group, including but not limited to pectin, gellan gum and soluble soybean polysaccharides. Other suitable polysaccharides include but are by no means limited to alginate and chitosan.

As will be appreciated by one of skill in the art, polysaccharides swell when they are in contact with water to form three dimensional networks. They help to sustain the physical and structural integrity of the particles. In particular, when the particles are subjected to acidic stomach conditions, the network formed by the carboxyl group-bearing polysaccharides in the particles contract, thereby helping to maintain the integrity of the particles, as discussed herein.

As will be appreciated by one of skill in the art, the starch source acts in the mixture as an absorbent of the essential oils. Specifically, when in liquid form, the oils can enter the pores of the starch granules. Incorporation into the starch granules provides better protection against absorption and/or degradation of the essential oils. In contrast, if the essential oils are added in a solid form, the essential oils are not absorbed and would essentially be only a part of the physical mixture of the components of the granulated particle, not absorbed and protected by the starch granules.

The starch source may be any suitable starch known in the art, for example, corn starch, wheat starch, potato starch, tapioca starch or the like. Alternatively, a modified starch such as maltodextrin may be used.

In some embodiments, the starch is a 1:1 to 3:1 mixture of corn starch and pre-gelatinized starch.

In some embodiments of the invention, a granulated particle according to the invention comprises:

5%-15% essential oil,

10%-15% fatty acid,

60%-80% starch source and

0.5-2% polysaccharide.

In some embodiments of the invention, a granulated particle according to the invention comprises:

10%-15% thymol,

10%-15% lauric acid,

60%-80% starch source and

0.5-2% polysaccharide.

Preferably, the granule size is between 5-20 microns. As will be apparent to one of skill in the art, granular particles of this size are known to distribute efficiently in feeds.

Preferably, the granulated particles should show a 60-80% stomach bypass rate and release kinetics of more than 95%. Mechanical strength of the granulated particles should be about 150-300 μN.

According to another aspect of the invention, there is provided a method of preparing a granulated particle comprising:

mixing an essential oil and a fatty acid;

adding a starch source to the mixture and mixing;

adding polysaccharide to the mixture;

allowing solid particles to form; and

granulating the particles.

The particles may be granulated by any means. In some embodiments, the particles are granulated using a granulating machine. In these embodiments, the granulating machine may have a membrane of between 0.3 mm-3 mm. As will be known by those of skill in the art, for feed applications ˜1 mm membranes are typically used.

In some embodiments, the fatty acid is a medium chain fatty acid, as discussed herein.

In some embodiments, the fatty acid is selected from the group consisting of monolaurin, caprylic acid, triglycerides of caprylic acid, capric acid, trigicerides of capric acid, and lauric acid.

In some embodiments, the essential oil is selected from the group consisting of carvacrol, cinnamaldehyde, thymol, citral, curcumin, allicin and capsaicin.

In some embodiments, the essential oil is thymol and the fatty acid is lauric acid.

Specifically, as discussed herein, thymol and lauric acid are very compatible. While note wishing to be bound to a particular theory or hypothesis, it is believed that this compatibility is due to the fact that thymol and lauric acid have a similar melting point. Alternatively, thymol may form hydrogen bonds with lauric acid more easily than with other fatty acids with more carbons. It is also possible that the mixture of thymol and lauric acid slows down crystallization rate.

The polysaccharides can be any polysaccharide that contains a carboxyl group, including but not limited to pectin, gellan gum and soluble soybean polysaccharides. Other suitable polysaccharides include but are by no means limited to alginate and chitosan.

As will be appreciated by one of skill in the art, polysaccharides swell when they are in contact with water to form three dimensional networks. They help to sustain the physical structure integrity of the particles. In particular, when the particles are in acidic stomach conditions, the network formed by the carboxyl group-bearing polysaccharides in the particles contract, thereby helping to maintain the integrity of the particles, as discussed herein.

As will be appreciated by one of skill in the art, the starch source acts in the mixture as an absorbent of the essential oils. Specifically, when in liquid form, the oils can enter the pores of the starch granules. Incorporation into the starch granules provides better protection against absorption and/or degradation of the essential oils. In contrast, if the essential oils are added in a solid form, the essential oils are not absorbed and would essentially be only a part of the physical mixture of the components of the granulated particle, not absorbed and protected by the starch granules.

The starch source may be any suitable starch known in the art, for example, corn starch, wheat starch, potato starch, tapioca starch or the like. Alternatively, a modified starch such as maltodextrin may be used.

In some embodiments, the starch is a 1:1 to 3:1 mixture of corn starch and pre-gelatinized starch.

In some embodiments, particle formation is promoted by placing the mixture in an ice bath. However, this is not essential as the material could also be stored at 0-4 C or even room temperature for particle formation.

In some embodiments of the invention, a granulated particle according to the invention comprises:

5%-15% essential oil,

10%-15% fatty acid,

60%-80% starch source and

0.5-2% polysaccharide.

Preferably, the granule size is between 5-20 microns. As will be apparent to one of skill in the art, granular particles of this size are known to distribute efficiently in feeds.

Preferably, the granulated particles should show a 60-80% stomach bypass rate and release kinetics of more than 95%. Mechanical strength of the granulated particles should be about 150-300 μN. As discussed herein, adoption of a fatty acid (such as lauric acid) serves two purposes: 1. act as a carrier for thymol; 2. provide an additive, if not synergistic, antibacterial effect with thymol; B. controlled and target release of thymol is achieved by using an economical and scalable encapsulation process; C. Lauric acid has significantly reduced the melting point of thymol which provides the convenience of processing thymol at room temperature (23° C.) in liquid form.

The formulation and method developed for encapsulation of thymol in solid granules are relatively simple and economical and can be used to deliver essential oils effectively to pig intestinal tract.

The invention will now be further elucidated and explained by way of examples; however, the invention is not necessarily limited to the examples.

### Example 1—Selection of a Fatty Acid Compatible with Thymol to Obtain Granules

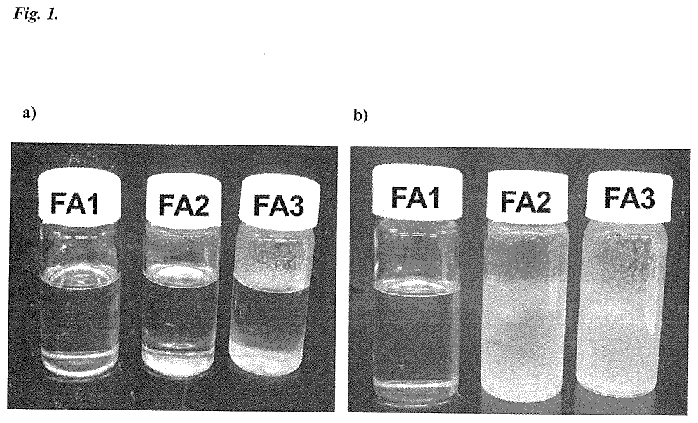

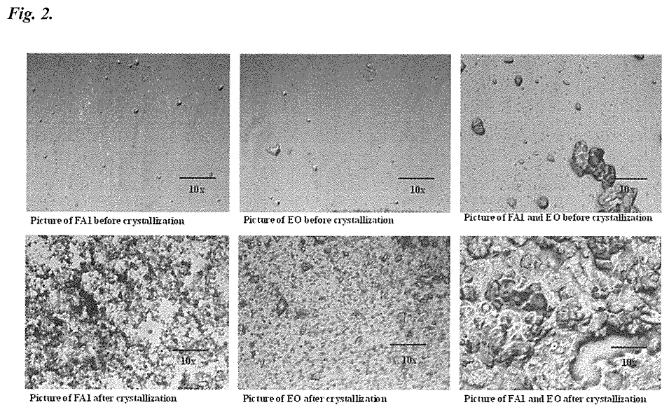

As shown in FIG. 1a, there was no phase separation observed in the mixtures between thymol and all three fatty acids at room temperature (23° C.) for 0 min. As shown in FIG. 1b, after 10 h at room temperature (23° C.) the molten mixture of thymol and lauric acid was in liquid state without having phase separation. However, the molten mixture of thymol and palmitic acid started to solidify and formed a gel-like mixture and the same happened to the molten mixture of thymol and stearic acid. As shown in FIG. 2, lauric acid was in the form of droplets with round shapes when observed at room temperature (23° C.) before solidifying and the shape was still uniform after solidifying. Thymol was in round shaped particles before solidifying and the shape of the particles changed to an irregular shape after solidifying. The thymol and lauric acid mixture showed that before solidifying the particles were together which was also evident after crystallization, as there was no form of separation between the two particles which would comprise of particles having irregular shapes and round shapes. The melting point of the thymol and lauric acid mixture was significantly lower than the melting points of the individual compound. These results indicated that lauric acid is a good carrier for thymol to form core granules.

The success of developing solid granules containing lipid and thymol depends on several factors that affect the granule properties including granule size and release kinetics. One of these factors is the compatibility of thymol and lipid (e.g. fatty acids) (Ma et al., 2016). In this study, lauric acid had the best compatibility with thymol compared to palmitic acid and stearic acid. While not wishing to be bound to a particular theory or hypothesis, it is believed that this compatibility is due to the fact that thymol and lauric acid have a similar melting point. Alternatively, thymol may form hydrogen bonds with lauric acid more easily than with other fatty acids with more carbons. It is also possible that the mixture of thymol and lauric acid slows down the crystallization rate. In addition, it has significantly reduced the melting point of thymol which provides the convenience of processing thymol at room temperature (23° C.) in liquid form.

An in vitro study demonstrated that Brachyspira hyodysenteriae, the causative pathogen of swine dysentery, was sensitive to lauric acid with minimum inhibitory concentration (MIC) values less than 1.5 mM (Vande Maele et al., 2016). Dietary fats rich in lauric acid and myristic acid increased broiler performance that may be related to both fatty acids' antimicrobial property (Zeitz et al., 2015). More recently a study showed that lauric acid can be used as a feed additive to reduce Campylobacter spp. levels in broiler meat (Zeiger et al., 2017). Lauric acid also has a strong antibacterial effect on gram positive bacteria, such as for example Streptococcus and Clostridia. Lauric acid has a positive effect on the intestinal integrity of animals and a positive effect on growth performance. Lauric acid is most suitable for pigs, poultry and calves. Lauric acid's derivatives (e.g. monolaurin) is known for its protective biological activities as an antimicrobial agent (Seleem et al., 2016). Therefore, in this study lauric acid is not only a carrier for thymol, but also a bioactive compound with antimicrobial properties.

### Example 2—Surface Diagram of Lipid Matrix Granules with/without 2% Polysaccharides Observed with a Light Microscope

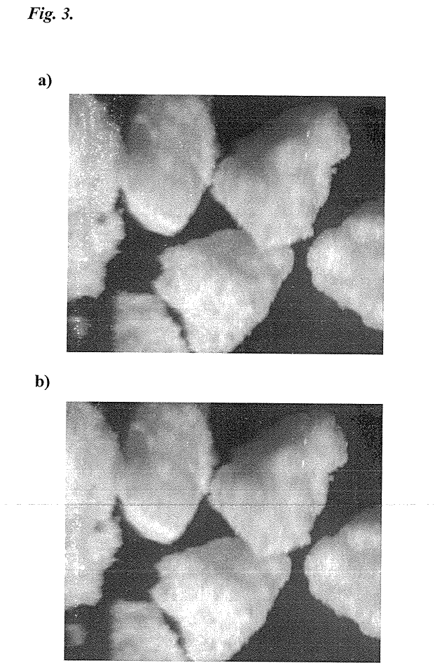

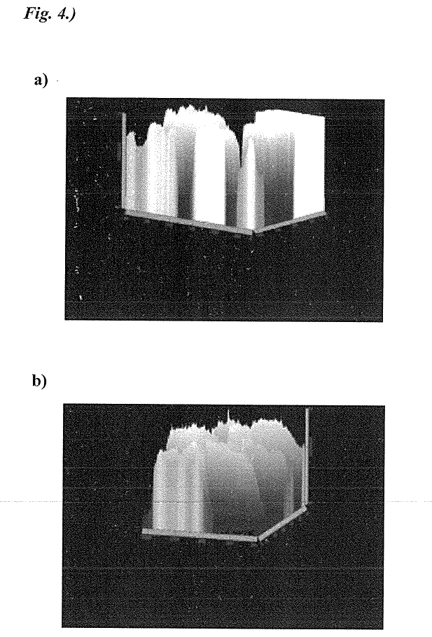

As shown in FIGS. 3 and 4, the diagram showing the outer surface of the picture revealed that there was a significant difference between the lipid matrix granules without polysaccharides when compared with lipid matrix granules with the addition of 2% polysaccharides. The surface of the lipid matrix granules without polysaccharides was coarse and had rough edges while the lipid matrix granules with the addition of 2% polysaccharide exhibited a smooth edge surface.

As will be appreciated by one of skill in the art, the smooth surface edge indicates the particles have a better sphericity and smaller specific surface area than the particles without added polysaccharide. As such, added polysaccharides such as alginate polysaccharide makes the particles more spherical and therefore more stable.

The composition of lipid matrix granules with polysaccharides incudes 66.22% corn starch, 11.03% pre-gelatinized starch, 11.03% thymol, 11.03% lauric acid and 0.7% alginate (polysaccharides). The average particle sizes of the lipid matrix granules were 12±2.69 μm in diameter.

A wide variety of natural and synthetic polymers have been used to enclose and protect bioactive agents. For applications in animal feeds, it is better to use natural polymers that have been approved for use in feeds. Alginate is a linear and anionic polysaccharide derived from brown seaweed and remains an attractive material for feed applications. It is composed of alternating block of α-1,4-]-guluronic acid (G) and β-1,4-d-manurunic acid (M) units (Dragan, 2014). Moreover, alginate is soluble in water at room temperature (23° C.), meaning that it does not require heating and cooling cycles for the formation of gels (Agüero et al., 2017). In this study, alginate improved spherical surface, which may be due to its remarkable crosslinking capability.

### Example 3—In Vitro Release Profile of Thymol and Lauric Acid from Lipid Matrix Granules with/without 2% Polysaccharide Using Simulated Fluids

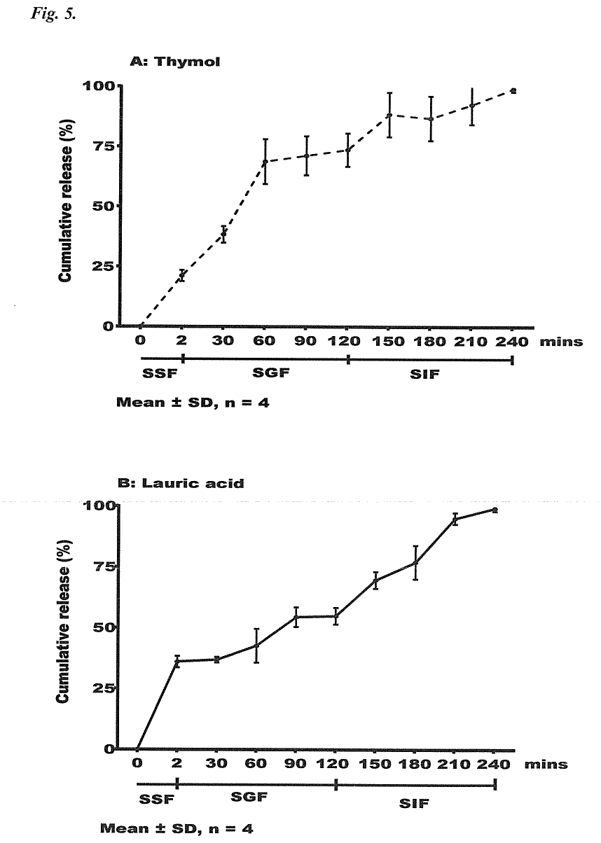

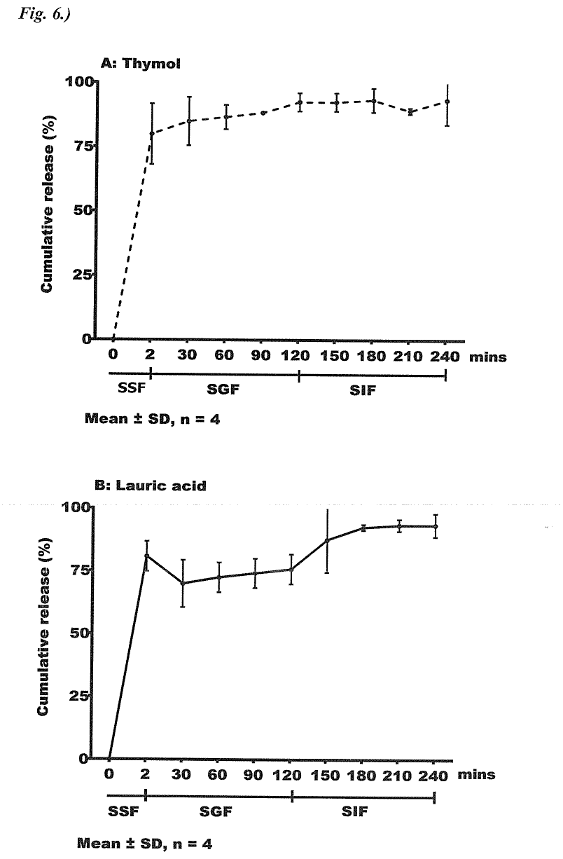

As shown in FIG. 5, the lipid matrix granules produced without 2% polysaccharide had quick release rates of thymol (79.9±11.8%) and lauric acid (80.8±5.9%) after incubated in the SSF for 2 min. When the lipid matrix granules were placed in the SGF for varying time intervals (30, 60, 80 and 120 min) respectively, the cumulative release (%) of thymol was 84.9±9.4, 86.6 4.7, 88.3±0 and 92.5±3.5, respectively. The cumulative release (%) of lauric acid was 69.9±9.4, 72.4±5.8, 74.1±5.8 and 75.8±5.9, respectively. Thymol and lauric acid were almost all released before getting to SIF and only few thymol (92.5±3.5; 93.3 4.6; 89.1±1.2; 93.3±9.4) % and lauric acid (87.4±12.9; 92.4±1.2; 93.3 2.4; 93.3±4.6) % were released in the SIF at 150, 180, 210 and 240 min respectively. However, as shown in FIG. 6 the lipid matrix granules with 2% polysaccharide exhibited a slow release (%) for thymol (21.2±2.3) and lauric acid (36±1.1) in the SSF. When the SGF was added, the cumulative release (%) of thymol (38.4±3.4; 68.8±9.3; 71.2±8.1; 73.7±6.9) and lauric acid (36.8±0.6; 42.6±3.4; 54.4±2.0; 54.8±1.7) were increased gradually at 30, 60, 80 and 120 min. In the SIF, the cumulative release (%) of thymol (88.4±9.2; 86.8±9.2; 92.5±8.1; 99.1±1.2) and lauric acid (69.6±1.7; 77±3.4; 95±1.1; 99.1±0.6) progressively increased at 150, 180, 210 and 240 min respectively until the time point was finally reached as shown in FIG. 6.

Alginate has pH-sensitivity because of the presence of carboxylic groups in the alginate structure. When subjected to a pH lower than its pKa (pH<3.4), the carboxylic acid groups are in a non-ionized form (COOH), leading to the formation of an insoluble structure (Agüero et al., 2017). At pH >4.4, the carboxylic group became inozied (COO—) resulting in an increase in electrostatic repulsion of these negative charges causing polymer chain expansion and swelling of the hydrophilic matrix. This effect is greatest around pH 7.4, which is similar to intestinal pH. In this study, the results clearly demonstrated that alginate significantly decreased the release of thymol and lauric acid in acidic environment and increased their release in the simulated intestinal fluids.

### Example 4—the Stability of Lipid Matrix Granules with/without 2% Polysaccharides During Storage

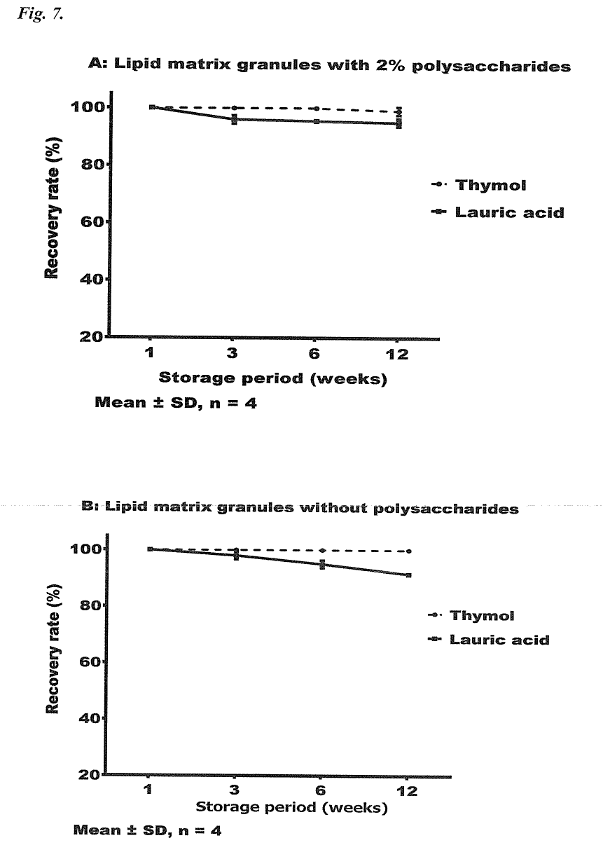

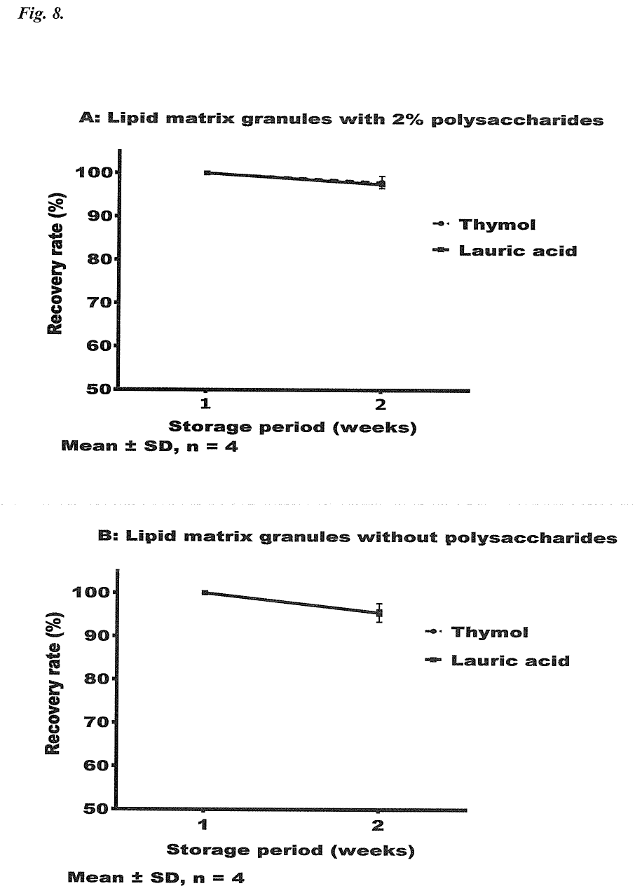

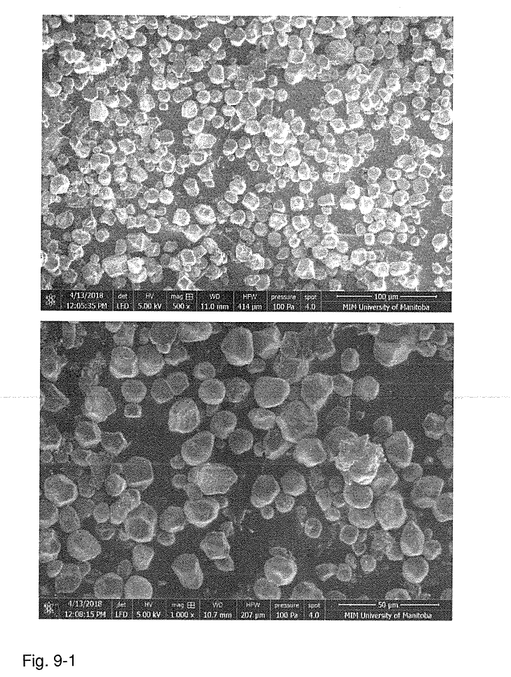

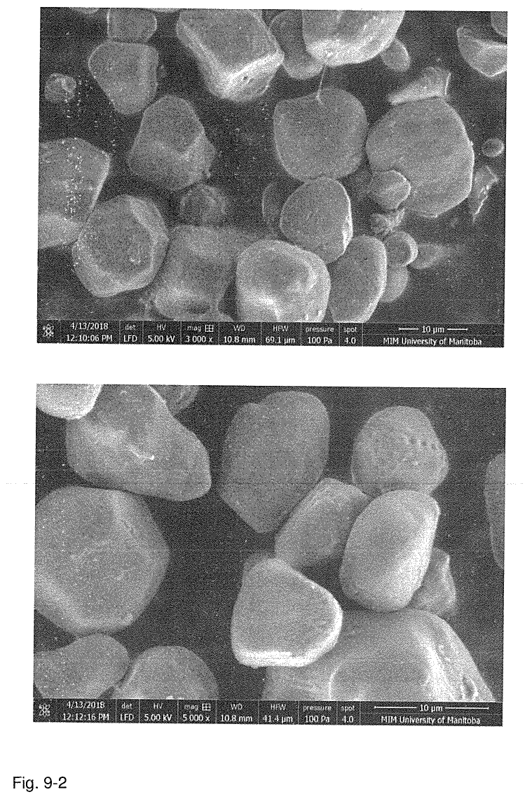

As shown in FIG. 7, the lipid matrix granules with or without polysaccharides both had a good stability (>90%) of both thymol and lauric acid after stored at 4° C. for 12 weeks. As shown in FIG. 8, the lipid matrix granules with or without polysaccharides both had a good stability (>95%) of thymol and lauric acid after stored at 4° C. at room temperature (23° C.) for 2 weeks.

Stability during storage is an important factor that needs to be considered for feed additives. Feed additives often must have a 1-2 year shelf life. Our preliminary data demonstrated that the lipid matrix granules are stable during short-term storage. Inclusion of antioxidants in the formula could be considered to improve stability of granules.

As will be apparent to one of skill in the art, any suitable antioxidant agent, for example, antioxidant compounds that are compatible with and suitable for use in animal feeds, may be used within the invention. Useful antioxidant agents include, for example, butylated hydroxyanisole (BHA), butylated hydroxytoluene (BHT), eugenol, ethoxyquin, propyl gallate, tertiary butyl hydroquinone, (TBHQ), tocopherols and the like. The antioxidant agents can be employed in the lipid matrix granules of the invention in amounts effective to increase the shelf life of the feed additives, for example by reducing the rate of rancidity conversion in the lipid matrix granules of the invention. Useful amounts of the antioxidant agents may range from about 100 to about 4000 ppm, more preferably from about 200 to about 2000 ppm, and most preferably from about 300 to about 1000 ppm, based on the quantity of fatty acids employed.

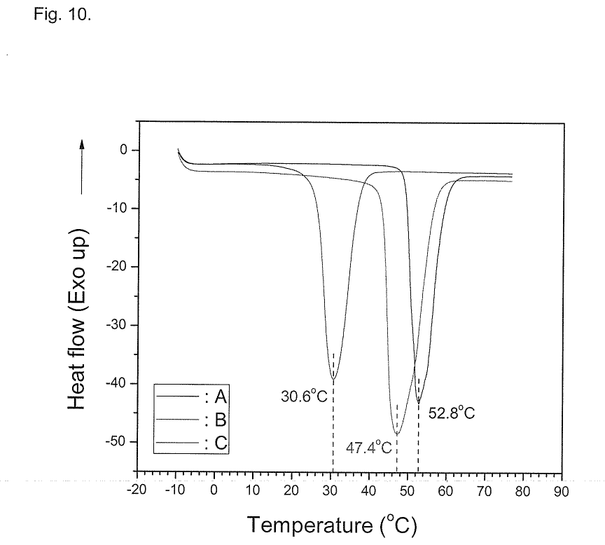

Lauric acid and thymol can make a eutectic mixture that is a mixture of two or more pure chemicals which usually do not interact to form a new chemical compound but, which at certain ratios, inhibit the crystallization process of one another resulting in a system having induced melting point depression. As shown in FIG. 10, the eutectic mixture of lauric acid and thymol has a melting point of 30.6° C. that is lower than either of thymol (52.8° C.) and lauric acid (47.4° C.).

### Example 5—Materials

Thymol (≥98.5%), lauric acid (LA, C₁₂), palmitic acid (PA, C₁₆), stearic acid (SA, C₁₈), amylase, pepsin from porcine gastric mucosa, pancreatin from porcine pancrease, corn starch, pre-gelatinized starch and alginate were obtained from Sigma-Aldrich (Oakville, Ontario, Canada).

### Example 6—Selection of Suitable Fatty Acid as a Carrier for Thymol

Lauric acid (LA, C₁₂), palmitic acid (PA, C₁₆) and stearic acid (SA, C₁₈) were used in this experiment because these three fatty acids have melting points above thymol's melting point (42° C.). The melting points of lauric acid, palmitic acid and stearic acid are 43° C. 63° C. and 69° C. respectively. 10 g of thymol was weighed in triplicate and put in three dishes (Pyrex® 190×100, No 3140, Germany). 10 g of each of the fatty acids was added to each dish, respectively and melted in a water bath at 70° C. After melting, the mixtures were stirred (IKA® C-MAG HS7) at level 1 with a stirring bar for 30 min. The moten mixture of each fatty acid with thymol was allowed to stand at 55° C. without stirring for 2 h before standing at room temperature (23° C.) for 6 h to allow for solidification. The morphology of thymol, fatty acids and the mixture thereof was observed with a light microscope at room temperature (23° C.). The materials were then placed in the refrigerator at 4° C. overnight to solidify. After solidification, a light microscope was also used to observe the morphology of thymol, fatty acids and the mixture thereof.

### Example 7—Preparation of Lipid Matrix Granules

For the first formulation, 5 g of lauric acid and thymol each were weighed into a closed vial separately and melted at 55° C. in a water bath, then mixed together and stirred with a stirring bar for 30 min. Corn starch and pre-gelatinized starch were weighed at ratio 3:1 (total 30 g) and hand stirred together in a container. The molten oil was mixed with the starch mixture by hand stirring before adding distilled water. The solid particles produced were immediately inserted into an ice-water bath for 1 h and 30 min and kept in the refrigerator at 4° C. overnight for further processing. The solid particles were granulated into micro-particles with a granulating machine (UAM Pharmag, Germany) at 90 rpm using a pore size of 0.1 mm and dried in room temperature (23° C.) for 1 h before storage at 4° C. For the second formulation, the procedures were similar to the first formulation, but the major difference was to replace the water with 2% alginate solution.

### Example 8—Morphology and Microstructure of Lipid Matrix Granules

The morphology and the structure of the lipid matrix granules produced were determined with a light microscope (Axio Cam 105, Carl-Zeiss, Switzerland; Nikon eclipse, Japan) and Zen Image Software (2012) was used to determine the surface diagram of the lipid matrix granules produced with or without 2% polysaccharides.

### Example 9—In Vitro Release of Thymol and Lauric Acid from the Lipid Matrix Granules

In vitro release of thymol and lauric acid from the lipid matrix granules was determined using previously published procedures (Minekus et al. 2014) with some modifications. Briefly, simulated salivary fluid (SSF) containing KCl, KH₂PO₄, NaHCO₃, MgCl₂(H₂O)₆ and (NH₄)₂CO₃, simulated gastric fluid (SGF) containing KCl, KH₂PO₄, NaHCO₃, NaCl, MgCl₂(H₂O)₆ and (NH₄)₂CO₃ and simulated intestinal fluid (SIF) containing KCl, KH₂PO₄, NaHCO₃, NaCl, MgCl₂(H₂O)₆ were prepared and their pH was adjusted to 7, 3 and 7 respectively with 0.1 M of HCl or 0.1 M of NaOH. The final digestion mixture of the electrolyte solution for simulated salivary fluid (SSF), simulated gastric fluid (SGF) and simulated intestinal fluid (SIF) was prepared by adding CaCl₂(H₂O)₂ to the mixture and then adding the respective digestive enzyme to simulate digestion in pig digesta. Specifically, Alpha-amylase from human saliva was added to the SSF digestion mixture, pepsin from porcine gastric mucosa was added to the SGF digestion mixture and pancreatin from porcine pancreas was added to the SIF digestion mixture and stirred for 30 min before testing. 36 samples were used to simulate pig digesta with 4 samples representing each time point. All solutions were maintained at 37° C. SSF was added to each of the samples at a ratio of 1:1 and placed in an incubator shaker (Innova™. 4200, New Brunswick Scientific, Edison/NJ. USA) for 2 min and the pH adjusted to 3 with 0.1 μl of HCl to stop enzymatic digestion. After that, SGF was added to the chyme and shaken in the incubator for 2 h; every 30 min, 4 samples were removed to represent each time point and the pH was adjusted with 0.2 μl of NaOH to stop peptic digestion followed by adding SIF to the chyme and shaken in the incubator for 2 h with 4 samples removed every 30 min representing each time point. 5 ml of oil extraction solvent (hexane) was added to each of the supernatants, which was then shaken (IKA Vibrax VXR Basic, U.S.A) for 20 min and allowed to stand for 30 min. Each of the supernatants from each point were diluted 10 times and the diluent was filtrated using a syringe-driven filter unit (polyetrafluoroethylene, 0.22 nm) and further analyzed by gas chromatography (GC) following the method explained herein. Two replicates for each sample were used.

The column installed was SUPELCO WAX™ 10 (fused silica capillary column; 60 m×0.25 mm×0.50 nm film thickness and the temperature limits from 35-280° C.). The injection volume was 1.0 nl. Post column length-2m and post column ID-0.363 mm. Oven set point-80° C., front PTV inlet-250° C., back s/SL inlet-57.5° C., back FID base-49.5 pA. Flow rate: front PTV carrier-2 ml/min, front PTV split-20 ml/min, back s/SL split-5.1/min. Pressure: front PTV carrier-239.46 kpa. Elapsed time-24 min, hold time-1 min. Split ratio—1:10. Stock solution for thymol and lauric acid was prepared separately by weighing 0.2 g into 10 ml of a volumetric flask and topped up with hexane. The standard solution was prepared by measuring 50 μl, 100 μl, 150 μl, 200 μl and 250 μl from the stock solution into a 5 ml volumetric flask and topped up with hexane using HPLC grade with their concentration ranging from 200 μg/ml to 1000 μg/ml. Each of the standards prepared was transferred into a capped GC-vial and run with other samples to determine their concentrations.

Thymol and lauric acid were identified by comparing the retention time with the standard thymol and lauric acid and their concentrations were calculated by comparing the total peak area of thymol and lauric acid with the standard curve (y-axis: thymol and lauric acid concentration: 200 μg/ml to 1000 μg/ml and X-axis: peak area). Thymol and lauric acid content=thymol or lauric acid concentration in GC vial×5(volume of added hexane for thymol/lauric acid concentration)×dilution times/weight of dry samples (˜0.5 g)×100%.

### Example 10—Determining Stability of the Lipid Matrix Granules

The stability of the lipid matrix granules with or without 2% polysaccharides was determined by storing the lipid matrix granules (n=4) at 4° C. for 12 weeks and then storing the granules at room temperature (23° C.) for 2 weeks. The lipid matrix granule samples taken from different time points were weighted in triplicates into a 15 ml glass vial. 1% of pancreatin from porcine pancrease was dissolved in distilled water and added to the weighed samples before placing the samples in the incubator. The samples were placed in an incubator shaker (Innova™. 4200, New Brunswick Scientific, Edison/NJ. SA) at 300 rpm at 37° C. for 30 min and allowed to stand for 20 min after which 5 ml of hexane (used as an extraction solvent) was added. The mixture was vortexed for 20 min at 1000 rpm and allowed to stand for 30 min before the oily phase was extracted and diluted 10 times with hexane and the diluent filtered with a syringe-driven filter unit (polyetrafluoroethylene, 0.22 nm) before being analyzed by GC following the method described below. Each of the samples was in triplicate. The total oil content in the microparticle was calculated using the formula below: EO and OA1 content in the lipid matrix granules=thymol/lauric acid concentration in GC vial×5 (volume of added hexane for thymol/lauric acid concentration)×dilution times/weight of dry samples (˜0.5 g)×100%.

### Example 11—Scanning Electron Microscope

Scanning electron microscope (SEM, FEI Quanta E-SEM) images of the micron-beads were captured at 5 kV without Au—Pd coating to evaluate diameters (DIs) of these beads.

### Example 12—Melting Point of Thymol, Lauric Acid, and Mixture of Thymol and Lauric Acid

1 g of thymol and 1 g of lauric acid was mixed by vortexing for 30 sec at 3,000 rpm, and then kept in −80° C. for 3 h. The mixture was ground to a fine powder using a grinder. Measurement of melting temperature was performed by differential scanning calorimetry (DSC). 10-15 mg of sample was weighed and added to a Tzero Aluminum hermetic pan. The pan was placed in the chamber of a DSC (Q Series DSC, TA Instrument). The DSC evaluated the melting temperature of samples according to the program: 1) Equilibrate at 25° C.; 2) Jump to −10° C.; 3) Ramp 10° C./min to 80° C. (1st 25 run); 5) Cooling; 6) Equilibrate at −10° C.; 7) Isothermal for 5 min; and 8) Ramp 10° C./min to 80° C. (2nd run).

The scope of the claims should not be limited by the preferred embodiments set forth in the examples but should be given the broadest interpretation consistent with the description as a whole.

### REFERENCES

1. Agüero L, Zaldivar-Silva D, Peña L, Dias M L. Alginate microparticles as oral colon drug delivery device: A review. Carbohydr Polyme. 2017, 168: 32-43.
2. Bedford M R, Cowieson A J. Exogenous enzymes and their effects on intestinal microbiology. Anim Feed Sci Technol. 2012, 173: 76-85.
3. Bengtsson B, Wierup M. Antimicrobial resistance in Scandinavia after ban of antimicrobial growth promoters. Anim Biochnol. 2006, 12: 147-156.
4. Boyen F, Haesebrouck F, Vanparys A, Volf J, Mahu M, Van Immerseel F, Rychlik I, Dewulf J, Ducatelle R, Pasmans F. Coated fatty acids alter virulence properties of Salmonella Typhimurium and decrease intestinal colonization of pigs. Vet Microbiol. 2008, 132: 319-327.
5. Brenes A, Roura E. Essential oils in poultry nutrition: Main effects and modes of action. Anim Feed Sci Tech. 2010, 158: 1-14.
6. Chen J, Wang Q, Liu C M, Gong J. Issues deserve attention in encapsulating probiotics: critical review of existing literatures. Crit Rev Food Sci and Nutr. 2017, 57: 1228-1238.
7. Chitprasert P, Sutaphanit P. Holy basil (Ocimum sanctum Linn.) essential oil delivery to swine gastrointestinal tract using gelatin microcapsules coated with aluminum carboxymethyl cellulose and beeswax. J Agric Food Chem. 2014, 62: 12641-12648.
8. Choi S C, Ingale S L, Kim J S, Park Y K, Kwon I K, Chae B J. An antimicrobial peptide-A3: Effects on growth performance, nutrient retention, intestinal and faecal microflora and intestinal morphology of broilers. Br Poult Sci. 2013, 54: 738-746.
9. Dragan E S. Design and applications of interpenetrating polymer network hydrogels. Chem Engin J. 2014, 243: 572-590.
10. De Lange C F M, Pluske J R, Gong J, Nyachoti C M. Strategic use of feed ingredients and feed additives to stimulate gut health and development in young pigs. Livest Sci. 2010, 134: 124-134.
11. Dierick N, Decuypere J, Molly K, Van Beek E, Vanderbeke E. The combined use of triacylglycerols containing medium-chain fatty acids (MCFAs) and exogenous lipolytic enzymes as an alternative for nutritional antibiotics in piglet nutrition: I. In vitro screening of the release of MCFAs from selected fat sources by selected exogenous lipolytic enzymes under simulated pig gastric conditions and their effects on the gut flora of piglets. Livest Prod Sci. 2002, 75: 129-142.
12. Eckel B, Kirchgessner M, Roth F X. Influence of formic acid on daily weight gain, feed intake, feed conversion rate and digestibility. J Anim Physiol Anim Nutr. 1992, 67: 93-100.
13. Gong J, Yin F, Hou Y, Yin Y. Chinese herbs as alternatives to antibiotics in feed for swine and poultry production: Potential and challenges in application. Can J Anim Sci. 2014, 94: 223-241.
14. Hanczakowska E, Szewczyk A, Swiatkiewicz M, Okon K. Short- and medium-chain fatty acids as a feed supplement for weaning and nursery pigs. Pol J Vet Sci. 2013, 16: 647-654.
15. Heo J M, Opapeju F O, Pluske J R, Kim J C, Hampson D J, Nyachoti C M. Gastrointestinal health and function in weaned pigs: a review of feeding strategies to control post-weaning diarrhoea without using in-feed antimicrobial compounds. J Anim Physiol Anim Nutr (Berl). 2013, 97: 207-237.
16. Hulánková R, Bořilová G. In vitro combined effect of oregano essential oil and caprylic acid against Salmonella serovars, Escherichia coli O157: H7, Staphylococcus aureus and Listeria monocytogenes. Acta Vet Brno. 2011, 80: 343-348.
17. Kim S W, Fan M Z, Applegate T J. Nonruminant nutrition symposium on natural phytobiotics for health of young animals and poultry: Mechanisms and application. J Anim Sci. 2008, 86: E138-139.
18. Lambert R J W, Skandamis P N, Coote P J, Nychas G J E. A study of the minimum inhibitory concentration and mode of action of oregano essential oil, thymol and carvacrol. Appl Microbiol. 2001, 91: 453-462.
19. Li S Y, Ru Y J, Liu M, Xu B, Peron A, Shi X G. The effect of essential oils on performance, immunity and gut microbial population in weaner pigs. Livest Sci. 2012, 145:119-123.
20. Ma Y H, Wang Q, Gong J and Wu X Y. Formulation of granules for site-specific delivery of an antimicrobial essential oil to the animal intestinal tract. J Pharm Sci. 2016, 105:1124-1133.
21. Michiels J, Missotten J, Dierick N, Fremaut D, Maene P, ade Smet S. In vitro degradation and in vivo passage kinetics of carvacrol, thymol, eugenol and trans-cinnamaldehyde along the gastrointestinal tract of piglets. J Sci Food Agric. 2008, 88:2371-2381.
22. Musa H H, Wu S L, Zhu C H, Seri H I, Zhu G Q. The potential benefits of probiotics in animal production and health. J Anim Vet Adv. 2009, 8: 313-321.
23. Piva A, Pizzamiglio V, Morlacchini M, Tedeschi M, Piva G. Lipid microencapsulation allows slow release of organic acids and natural identical flavors along the swine intestine. J Anim Sci. 2007, 85: 486-493.
24. Puvaca N, Stanacev V, Glamocic D, Levicc J, Peric L, Stanacev V, Milic D. Beneficial effects of phytoadditives in broiler nutrition. World's Poult Sci J. 2013, 69:27-34.
25. Randrianarivelo R, Danthu P, Benoit C, Ruez P, Raherimandimby M, Starter S. Novel alternative to antibiotics in shrimp hatchery: Effects of the essential oil of Cinnamosma fragrans on survival and bacterial concentration of Penaeus monodon larvae. J Appl Microbiol. 2010, 109: 642-650.
26. Rossi R, Pastorelli G, Cannata S, Corino C. Recent advances in the use of fatty acids as supplements in pig diets: a review. Anim Feed Sci Technol. 2010, 162: 1-11.
27. Seleem D, Chen E, Benso B, Pardi V, Murata R M. In vitro evaluation of antifungal activity of monolaurin against Candida albicans biofilms. PeerJ. 2016, 4:e2148.
28. Si W, Gong J, Chanas C, Cui S, Yu H, Caballero C, Friendship R M. In vitro assessment of antimicrobial activity of carvacrol, thymol and cinnamaldehyde towards Salmonella serotype Typhimurium DT104: Effects of pig diets and emulsification in hydrocolloids. J Appl Microbiol. 2006, 101: 1282-1291.
29. Solis de Los Santos F, Donoghue A M, Venkitanarayanan K, Dirain M L, Reyes-Herrera I, Blore P J, Donoghue D J. Caprylic acid supplemented in feed reduces enteric Campylobacter jejuni colonization in ten-day-old broiler chickens. Poult Sci. 2008, 87:800-804.
30. Van Boeckel T P, Brower C, Gilbert M, Grenfell B T, Levin S A, Robinson T P, Teillant A, Laxminarayan R. Global trends in antimicrobial use in food animals. Proc Natl Acad Sci. 2015, 112: 5649-5654.
31. Vande Maele L, Heyndrickx M, Maes D, De Pauw N, Mahu M, Verlinden M, Haesebrouck F, Martel A, Pasmans F, Boyen F. In vitro susceptibility of Brachyspira hyodysebteruae to organic acids and essential oil components. J Vet Med Sci, 2016, 78: 325-328.
32. Windisch W, Schedle K, Plitzer C, Kroismayr A. Use of phytogenic products as feed additives for swine and poultry. J Anim Sci. 2008, 86: E140-E148.
33. Yang C B, Chowdhury M A K, Hou Y, Gong J. Phytogenic compounds as alternatives to in-feed antibiotics: potentials and challenges in application. Pathogens. 2015, 4: 137-156.
34. Zeiger K, Popp J, Becker A, Hankel J, Visscher C, Klein G, Meemken D. Lauric acid as feed additive—An approach to reducing Campylobacter spp. in broiler meat. PLoS One. 2017, 12(4):e0175693.
35. Zeitz J O, Fennhoff J, Kluge H, Stangl G I, Eder K. Effects of dietary fats rich in lauric and myristic acid on performance, intestinal morphology, gut microbes, and meat quality in broilers. Poult Sci. 2015, 94:2404-13.
36. Zentek J, Buchheit-Renko S, Ferrara F, Vahjen W, Van Kessel A, Pieper R. Nutritional and physiological role of medium-chain triglycerides and medium-chain fatty acids in piglets. Anim Health Res Rev. 2011, 12:83-93.
37. Zentek J, Buchheit-Ronko S, Munnor K, Piopor R, Vahjon W. Intestinal concentrations of free and encapsulated dietary medium-chain fatty acids and effects on gastric microbial ecology and bacterial metabolic products in the digestive tract of piglets. Arch Anim Nutr. 2012, 66:14-26.
38. Zhao J, Harper A F, Estienne M J, Webb K E Jr, McElroy A P, Denbow D M. Growth performance and intestinal morphology responses in early weaned pigs to supplementation of antibiotic-free diets with an organic copper complex and spray-dried plasma protein in sanitary and nonsanitary environments. J Anim Sci. 2007, 85:1302-1310.
39. Zhang Y, Wang Q C, Yu H, Zhu J, de Lange K, Yin Y, Wang Q, Gong J. Evaluation of alginate-whey protein microcapsules for intestinal delivery of lipophilic compounds in pigs. J Sci Food Agric. 2016, 96: 2674-2681.

1. A granulated particle comprising:
an essential oil;
a fatty acid or glyceride of a fatty acid;
a starch source; and
a polysaccharide.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. A method of preparing a granulated particle comprising:
mixing an essential oil and a fatty acid;
adding a starch source to the mixture and mixing;
adding polysaccharide to the mixture;
allowing solid particles to form; and
granulating the particles.

24. The method according to claim 23 wherein the fatty acid is a saturated fatty acid or glyceride thereof that is compatible with and suitable for use in animal feeds.

25. The method according to claim 23 wherein the fatty acid is a fatty acid that is solid at room temperature but forms a homogeneous liquid or liquid phase when mixed with the essential oil.

26. The method according to claim 23 wherein the fatty acid is a medium chain fatty acid.

27. The method according to claim 23 wherein the fatty acid is selected from the group consisting of monolaurin, caprylic acid, triglycerides of caprylic acid, capric acid, triglycerides of capric acid, and lauric acid.

28. The method according to claim 23 wherein the essential oil is selected from the group consisting of carvacrol, cinnamaldehyde, thymol, citral, curcumin, allicin and capsaicin.

29. The method according to claim 23 wherein the fatty acid is lauric acid and the essential oil is thymol, carvacrol or cinnamaldehyde.

30. The method according to claim 23 wherein the fatty acid is lauric acid and the essential oil is thymol.

31. The method according to claim 23 wherein the starch source is pre-gelatinized starch.

32. The method according to claim 23 wherein the starch source comprises pre-gelatinized starch.

33. The method according to claim 23 wherein the starch source is a mixture of pre-gelatinized starch and another starch.

34. The method according to claim 33 wherein the other starch is selected from the group consisting of corn starch, wheat starch, potato starch, tapioca starch and maltodextrin.

35. The method according to claim 33 wherein the starch source is a mixture of pre-gelatinized starch and corn starch.

36. The method according to claim 35 wherein the mixture of pre-gelatinized starch and corn starch is at a ratio of between 1:1 and 1:3.

37. The method according to claim 33 wherein the polysaccharide is a carboxyl group-containing polysaccharide.

38. The method according to claim 37 wherein the polysaccharide is selected from the group consisting of pectin, gellan gum, soluble soybean polysaccharides, alginate and chitosan.

39. The method according to claim 23 wherein the granulated particle comprises:
5%-15% essential oil,
10%-15% fatty acid,
60%-80% starch source and
0.5-2% polysaccharide.

40. The method according to claim 23 wherein the granulated particle:
10%-15% thymol,
10%-15% lauric acid,
60%-80% starch source and
0.5-2% polysaccharide.

41. The method according to claim 23 wherein the granulated particle size is between 5-20 microns.

42. The method according to claim 23 wherein the granulated particle has a 60-80% stomach bypass rate.

43. The method according to claim 23 wherein the granulated particle has release kinetics of more than 95%.

44. The method according to claim 23 wherein the granulated particle has a mechanical strength of between 150-300 μN.
